(19) United States
(12) Patent Application Publication
(10) Pub. No.: US 2012/0316594 A1
(43) Pub. Date: Dec. 13, 2012

(54) APPARATUS FOR CLOSING AN OPENING, SUCH AS A TROCAR OPENING, IN A PATIENT'S BODY

(75) Inventor: Michael Palese, New York, NY (US)

(73) Assignee: Mount Sinai School of Medicine, New York, NY (US)

(21) Appl. No.: 13/489,969

(22) Filed: Jun. 6, 2012

Related U.S. Application Data
(60) Provisional application No. 61/495,592, filed on Jun. 10, 2011.

(51) Int. Cl. A61B 17/03; A61B 17/34

(52) U.S. Cl. 606/185; 606/215

(57) ABSTRACT

A device for closing an opening formed in a patient's body without the use of sutures. The device includes a patch formed of a flexible that can be positioned between an open position and an at least partially closed position. The body has a plurality of sharp protrusions formed along the first face for securely attaching the flexible body to a target structure within the patient's body in which the opening is formed such that the flexible body extends across and closes the opening. The device further includes an instrument for delivering the patch to an inner face of a structure in which the opening is formed. The instrument has a first member that mates with the patch for securely, yet releasably, attaching the patch to the instrument to allow delivery of the patch.

### CROSS REFERENCE TO RELATED APPLICATION

The present application claims priority to and the benefit of U.S. Patent Application No. 61/495,592, filed Jun. 10, 2011 which is hereby incorporated by reference in its entirety.

### TECHNICAL FIELD

The present invention relates to medical equipment and more particularly to a device for closing a trocar site, without the use of sutures, after a surgical procedure, such as during a minimally invasive surgical procedure (e.g., a laparoscopic, robotic, endoscopic or arthroscopic procedure or NOS (natural orifice surgery) or NOTES (natural orifice transluminal endoscopic surgery).

### BACKGROUND

As technology advances, the manner in which surgical procedures are conducted and the instruments/tools that are used have greatly changed the field of minimally invasive surgery and has spawned many types of surgical procedures including laparoscopic, robotic, endoscopic and arthroscopic surgery. Laparoscopy (laparoscopic surgery) is an operation performed in the chest, abdomen, pelvis, or retroperitoeneum through small incisions (usually 0.5-1.5 cm) with the aid of a camera. It can either be used to inspect and diagnose a condition or to perform surgery. The laparoscope allows doctors to perform both minor and complex surgeries with a few small cuts in the abdomen. There are a number of advantages to the patient with laparoscopic surgery versus an open procedure. These include reduced pain due to smaller incisions and shorter recovery time. Robotic surgery is the latest advance in minimally invasive surgery which is similar to laparoscopic surgery in that surgery is performed through small incisions in the chest, abdomen, pelvis or retroperitoneum but uses a robotic system to perform surgery.

Endoscopic surgery is any surgery through a scope or instrument into a cavity such as endoscopy in the upper gastro-intestinal tract or cystoscopy in the lower genitor-urinary tract. This is a minimally invasive procedure that allows for the examination and treatment of tissues, organs, etc with a camera and working channel.

Arthroscopic surgery is related to a joint, while laparoscopic surgery is related to the abdominal cavity. Arthroscopy (also called arthroscopic surgery) is a minimally invasive surgical procedure in which an examination and sometimes treatment of damage of the interior of a joint is performed using an arthroscope, a type of endoscope that is inserted into the joint through a small incision. Arthroscopic procedures can be performed either to evaluate or to treat many orthopaedic conditions including torn floating cartilage, torn surface cartilage, ACL reconstruction, and trimming damaged cartilage.

Natural orifice surgery (NOS) describes scarless surgical techniques in which a natural orifice of the body is used to pass surgical instruments to the desired organ. The benefits of NOS are that the patient experiences significantly less pain, quicker recoveries, fewer complications and no scars. Natural orifice transluminal endoscopic surgery (NOTES) is a surgical technique whereby “scarless” abdominal operations can be performed with an endoscope based through a natural orifice (mouth, urethra, anus, etc.) then through an internal incision in the desired organ, such as the stomach, vagina, bladder, or colon, thus avoiding any external incisions or scars. By avoiding major incisions through the skin, muscle, and nerves of the abdomen, patients may experience a quicker recovery with less pain and scarring while reducing the risk of post operative hernias.

While technology has advanced and incision sizes have decreased, there is still a need to close the trocar site without using sutures. Currently after placement of a trocar for any laparoscopic or robotic procedure, the trocar site needs to be closed to avoid complications and in particular to avoid a possible trocar site hernia. In general, the larger trocars of 10 mm or greater in adult cases all trocars in pediatric cases require closure of the trocar site. This is usually done with placement of a suture into the fascia and peritoneal level. More specifically, in adult surgery, trocar sites are usually closed by single suturing of the peritoneum and deep fascia with absorbable or non-absorbable sutures, whereas the skin is sutured separately with absorbable or non-absorbable sutures.

Placement of the suture can be done under direct vision or with the use of a device that places the suture. While there are number of products available for suturing the trocar site, these products are suture based products that typically include mechanisms for delivering a needle and suture for closing an incision.

Placement of this trocar site suture can sometimes be tedious and difficult, adding time to the operative procedure. In addition, many patients complain of pain and discomfort at the closure site secondary to the tightness of the suture. There are also cases of nerve entrapment with closure of the trocar site.

It is therefore desirable to provide an apparatus and method for rapid closure of the trocar (port) site without the use of sutures.

### SUMMARY

In one embodiment of the present invention, a device for closing an opening formed in a patient's body without the use of sutures. The device includes a patch formed of a flexible body having a first face. The body is foldable and can be positioned between an open position and an at least partially closed position. The body has a plurality of sharp protrusions formed along the first face for securely attaching the flexible body to a target structure within the patient's body in which the opening is formed such that the flexible body extends across and closes the opening. The device further includes an instrument for delivering the patch to an inner face of a structure in which the opening is formed. The instrument has a first member that mates with the patch for securely, yet releasably, attaching the patch to the instrument to allow delivery of the patch.

In another embodiment, a device for closing a trocar site without the use of sutures includes a patch formed of a flexible body having a first face. The body is foldable and can be positioned between an open position and an at least partially closed position. The body has a plurality of sharp protrusions formed along the first face for securely attaching the flexible body to a target structure in which a trocar opening is formed such that the flexible body closes the trocar opening. The device also includes an instrument for delivering the patch to the trocar site. The instrument has a first coupling member that mates with the patch for securely, yet releasably, attaching the patch to the instrument to allow delivery of the patch.

The instrument and the patch are configured to be received within and be advanced through a hollow trocar for delivery to the trocar site for closing of the trocar opening due to the sharp protrusions being configured to be at least partially embedded within the target structure.

In yet another embodiment, a device for closing a trocar site without the use of sutures includes a patch formed of a flexible body having a first face. The body is foldable and can be positioned between an open position and an at least partially closed position. The body has an adhesive disposed along the first face for securely attaching the flexible body to a target structure in which a trocar opening is formed such that the flexible body closes the trocar opening.

In yet another embodiment, the body of the patch is formed of a biocompatible material that has both material characteristics and a modified surface structure that promotes attachment between the body and the target structure (tissue). For example, the patch body can be formed to include nanoscale hills and valleys on its surface, similar to the flexible nanopillars covering geckos' sticky toes so as to produce a “biorubbery” type body. The hills and valleys create a patterned interface that enhances the surface area of contact and thus, the overall strength of the adhesion. The surface area of the patch body can be coated with a material that promotes a stronger bond between select types of target structures and in particular, a thin layer of material (e.g., sugar-based adhesive material) can be added to enable a stronger bond to wet surfaces, such as the bladder, etc. When the patch body is applied, capillary forces pull tissue into spaces between the pillars and the glue adheres to tissue proteins. The patch can be biodegradable and dissolved over time. Even without the added adhesive material, capillary forces pull the tissue into spaces between the pillars resulting in attachment of the patch to the target structure (tissue).

The device also includes an instrument for delivering the patch to the trocar site. The instrument has a first coupling member that mates with the patch for securely, yet releasably, attaching the patch to the instrument to allow delivery of the patch. The instrument and the patch are configured to be received within and be advanced through a hollow trocar for delivery to the trocar site for closing of the trocar opening due to intimate contact between the adhesive and the target structure.

In another embodiment, a method for closing an opening formed in a patient's body comprising the steps of: inserting a trocar through an incision that defines a trocar opening for placement of the trocar; attaching a patch to a distal end of an instrument, the patch being formed of a flexible body having a first face, the body being foldable and can be positioned between an open position and an at least partially closed position, wherein the body has a plurality of sharp protrusions formed along the first face; inserting the folded patch and instrument within a bore of the trocar; advancing the patch and instrument within the bore of the trocar until the patch advances beyond a distal end of the trocar resulting in the folded patch at least partially opening, the first face of the patch facing an inner surface of a target structure through which the incision is formed; retracting the instrument in a proximal direction away from the patient's body so as to draw the patch into contact with the inner surface of the target structure, whereby the protrusions are at least partially embedded within the inner surface of the target structure, thereby securely attaching the patch thereto such that it closes off the trocar opening along the inner surface of the target structure; and disengaging the instrument from the patch, thereby leaving the patch in place covering the trocar opening.

These and other aspects, features and advantages shall be apparent from the accompanying drawings and description of certain embodiments of the invention.

### BRIEF DESCRIPTION OF THE DRAWINGS

FIG. 1 is a side perspective view of a device, according to a first embodiment, for closing a trocar site without sutures;

FIG. 2 is a perspective view, in an exploded view, of first and second components;

FIG. 3 is a partial perspective view of a portion of the first component which is in the form of a body that closes off the trocar site;

FIG. 4 is a side perspective view, in partial cross-section, of the device of FIG. 1, in an unassembled state and prior to insertion of the device into a bore of the trocar, which is shown inserted into an abdominal cavity;

FIG. 5 is a side perspective view of a second step of inserting the device of FIG. 1 into the bore of the trocar in which the first component is at least partially folded;

FIG. 6 is a side perspective view of a third step of delivering the device of FIG. 1 through the trocar to the trocar site (e.g., abdominal cavity);

FIG. 7 is a side perspective view showing a fourth step in which the first component unfolds after the first component is advanced beyond the distal end of the trocar;

FIG. 8 is a side perspective view showing a fifth step in which the trocar and a second component of the device are retracted from the body so as to seat the unfolded first component against a target structure, such as the peritoneum;

FIG. 9 is a cross-sectional view of the first component being securely attached to the target structure (e.g., peritoneum) with protrusions (e.g., teeth or barbs) being at least partially embedded therein;

FIG. 10 is a perspective view, in partial cross-section, showing the first component securely attached to the target structure (e.g., peritoneum) and the second component and trocar fully removed from the body, with a suture closing the incision along the skin surface;

FIG. 11 is a top perspective view of an alternative shape of the first component;

FIG. 12 is a top perspective view of an alternative shape of the first component;

FIG. 13 is a top perspective view of an alternative shape of the first component;

FIG. 14 is an exploded perspective view of a device, according to a second embodiment, for closing a trocar site without sutures;

FIG. 15 is an exploded perspective view of a device, according to a third embodiment, for closing a trocar site without sutures;

FIG. 16 is an exploded perspective view of a device, according to a fourth embodiment, for closing a trocar site without sutures;

FIG. 17 is a cross-sectional view of a second component of the device of FIG. 16 taken along the line 17-17 showing teeth embedded into a first component that closes the trocar entry site;

FIG. 18 is a cross-sectional view of the second component of the device of FIG. 16 with teeth embedded by disengaged from the first component by means of a pusher;

FIG. 19 is an exploded cross-sectional view of a device, according to a fifth embodiment, for closing a trocar site without sutures, showing an inner member in a retracted position;

FIG. 20 is a cross-sectional view showing the device of FIG. 19 with the inner member in an extended position and in intimate and secure attachment to a first component;

FIG. 21 is a cross-sectional view of the FIG. 19 with the inner member being retracted and disengaged from the first component;

FIG. 22 is an exploded perspective view of a device, according to a sixth embodiment, for closing a trocar site without sutures;

FIG. 23 is an exploded perspective view of a device, according to a seventh embodiment, for closing a trocar site without sutures;

FIG. 24 is an exploded perspective view of a device, according to an eighth embodiment, for closing a trocar site without sutures;

FIG. 25 is an exploded perspective view of a device, according to a ninth embodiment, for closing a trocar site without sutures, showing deployable fingers in a retracted position;

FIG. 26 is a perspective view of the first and second components mated together showing the deployable fingers in an open position to as to securely attach the first component to the second component;

FIG. 27 is a perspective view of a device according to a tenth embodiment, for closing a trocar site without sutures;

FIG. 28 is close-up partial perspective view of deployable struts in an open position so as to at least partially open a first component;

FIG. 29 is a close-up partial perspective view of the first component seated against the target structure (e.g., peritoneum) and the second component and trocar being removed from the body through the incision;

FIG. 30 is top perspective view of a device according to an eleventh embodiment, for closing a trocar site without sutures;

FIG. 31 is a cross-sectional view of the device of FIG. 30 in a fully extended position; and

FIG. 32 is a cross-sectional view of the device of FIG. 30 in a partially retracted position.

### DETAILED DESCRIPTION OF CERTAIN EMBODIMENTS OF THE INVENTION

The devices of the present invention have widespread use and are intended to be used in a number of different types of surgical procedures including, but not limited to, laparoscopic, robotic, endoscopic, arthroscopic surgery, NOS (natural orifice surgery), NOTES (natural transluminal endoscopic surgery) etc.

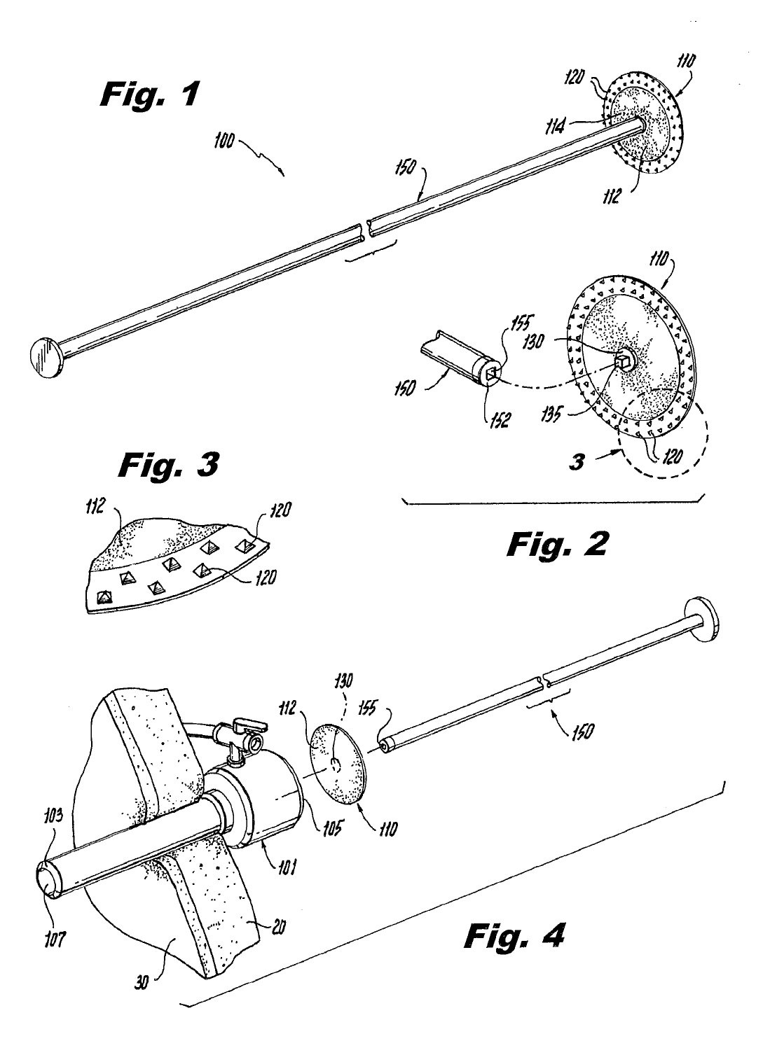

For purposes of illustration only, FIGS. 1-10 illustrate the application of the present invention in laparoscopic environment; however, this is merely one exemplary embodiment and, as described above, the devices of the present invention can equally be implemented in other types of surgical procedures including those described above.

As is well known, the abdominal cavity is the body cavity of the human body that holds the bulk of the viscera. It is located below (or inferior to) the thoracic cavity, and above the pelvic cavity. It is part of the abdominopelvic cavity. Organs of the abdominal cavity include the stomach, liver, gallbladder, spleen, pancreas, small intestine, kidneys and large intestine. The abdominal cavity is lined with a protective membrane termed the peritoneum. The kidneys are located in the abdominal cavity behind the peritoneum, in the retroperitoneum. The viscera are also covered, in the front, with a layer of peritoneum called the greater omentum (or omental apron).

A fascia is a layer of fibrous tissue that permeates the human body. A fascia is generally described as being connective tissue that surrounds muscles, groups of muscles, blood vessels, and nerves, binding those structures together. Fascia consists of several layer, namely, a superficial fascia, a deep fascia, and a subserous (or visceral) fascia and extends uninterrupted from the head to the tip of the toes.

In FIGS. 1-10, an abdominal cavity 10 is generally shown including fascia 20 and the peritoneum 30. As described above, the peritoneum 30 is a protective membrane that lines the abdominal cavity 10, with the fascia 20 overlying the peritoneum 30. It will be appreciated that when making an incision, as when introducing a trocar, the incision is made through the fascia 20 and then through the underlying peritoneum 30 to gain entrance into the abdominal cavity 10.

As is known in the industry, a trocar is a medical instrument with a sharply pointed end, often three-sided, that is used inside a hollow cylinder (cannula) to introduce the trocar into blood vessels or body cavities. Trocars are also used to introduce ports in the abdomen, such as during laparoscopic surgery. Trocar sites are the small entry sites made though abdomen for the entry of surgical instruments. After a small incision is made in the skin, the trocar is the instrument inserted to penetrate the abdominal wall. Trocars of different sizes can create entry sites ranging from 5 to 20 mm in diameter. The diameter size depends on whether the removal of a specimen is anticipated and its size and other considerations, such as the size/age of the patient. In conventional laparoscopic surgery, 3-5 ports are typically used; however, single port laparoscopy (also known as “single port surgery”) is the newest frontier in laparoscopic surgery. Laparoscopic surgery involves inflating the abdomen with an inert gas (CO₂) and performing an operation seen through a thin camera tube along with several long thin instruments inserted through separate “ports” or trocars. Single port laparoscopy uses just one port buried in the belly button to accommodate both the instruments and the camera. This would eliminate the use of up to 3 to 5 separate trocars for the performance of typical laparoscopic procedures and would potentially leave the patient with no visible scars. Despite the fact that the incisions are small, the trocar site still needs to be closed after the procedure as described above.

FIGS. 1-10 illustrate a trocar/port 101 that is inserted into the patient to provide access to the surgical site, such as a body cavity (e.g., the abdominal cavity 10). The trocar 101 has a first end 103 and an opposite end 105 that remains outside of the patient. A bore 107 is formed through the trocar 101 that permits instruments to be passed therethrough to the surgical site, such as a body cavity.

In accordance with the present invention, a number of different devices are provided and configured for closing a trocar site without the use of sutures. The devices are configured such that each one can be passed through the bore 107 of the trocar 101 and delivered to the trocar site to permit closure thereof as described below.

FIGS. 1-10 show a device 100 according to a first embodiment for closing a trocar site without the use of sutures. The device 100 is formed of two separate components, namely, a first component 110 that is configured to remain at the surgical site and close the trocar site and a second component 150 that is an applicator or delivery device for delivering the first component 110 to the trocar site. The first component 110 can thus be generally thought of as being a patch-like member that covers and closes the trocar site without the use of sutures.

In the illustrated embodiment, the first component 110 is defined by a body 112 that can be formed to have any number of different shapes and is sized in view of the application and the instruments used (i.e., in view of the specifications of the trocar itself). The body 112 has a first face or surface 114 and an opposing second face or surface 116. The shape and dimensions of the body 112 are such that when placed over the trocar entry site, the body 112 at least substantially and preferably covers the entire trocar entry site and even extends radially beyond the trocar entry site so as to cover a periphery around the trocar entry site as shown in FIG. 10.

In the illustrated embodiment, the first face 114 is the surface that faces the trocar entry site (and thus faces the target structure in which the trocar entry site is located). As described herein, the first face 114 has a contoured or modified surface that encourages an intimate engagement between the body 112 and the target structure in which the trocar entry site is located. For purposes of illustration, the target structure can be the peritoneum in the case of a laparoscopic technique as shown in FIGS. 4-10. FIGS. 4-10 show the abdominal cavity 10 and the fascia 20 and the underlying peritoneum 30. However, as mentioned above, the target structure can be some other tissue or membrane structure.

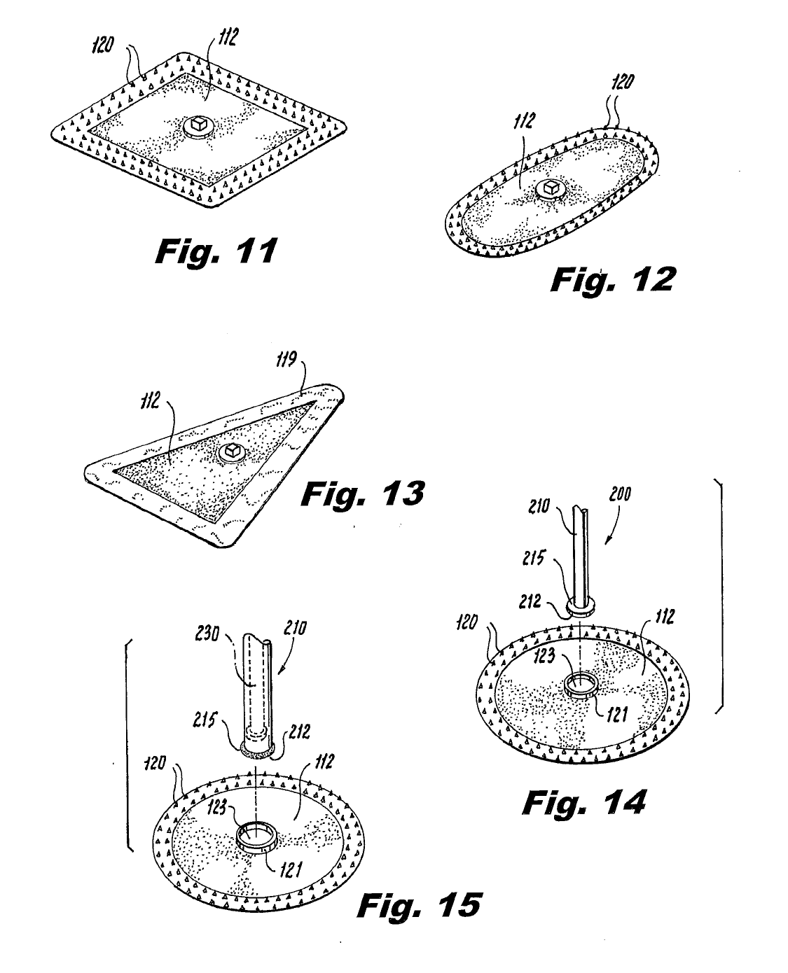

The body 112 is thus in the form of a flexible structure that can be freely folded and open. The body 112 can thus be thought of as being a patch that closes off the trocar entry site (an incision) by being attached across the incision. In a fully opened (flat) position, the body 112 can be generally planar in nature so as to permit the body 112 to seat against the target structure (e.g., the peritoneum). As mentioned above, the body 112 can be formed to have any number of different shapes, including but not limited to a circle, oval, square, rectangle, triangle, an irregular shape, etc. FIGS. 1-10 show the body 112 as being circular shaped. FIGS. 11-13 show alternative shapes for the body 112.

The first component 110 can be formed of any number of different materials that are suitable for the intended use and provide a biocompatible material that closes the trocar site. The first component 110 can be formed either of an absorbable material or it can be formed of a non-absorbable material.

For example, in one embodiment, the first component 110 is a synthetic absorbable body that is made primarily of polyglycolic acid. For example, the body can be formed at least partially of Vicryl (polyglactin 910) which is an absorbable, synthetic, braided suture, that is manufactured by Ethicon, Inc. The Vicryl suture holds its tensile strength for approximately three to four weeks in tissue, and is completely absorbed by hydrolysis within 60 days. Vicryl and other polyglycolic acid sutures can also be treated for more rapid breakdown in rapidly healing tissues or can be impregnated with triclosan to provide antimicrobial protection of the suture line. Although, the name “Vicryl” is a trademark of Ethicon, Inc., there are other commercial sources of synthetic absorbable sutures made primarily of polyglycolic acid. Other brands of polyglycolic acid suture include Surgicryl, Biovek, Visorb, Polysorb and Dexon, all of which are manufactured by different companies.

Absorbable first component 110 can be formed either of “natural” or synthetic polymers. Synthetic polymers are absorbed by hydrolysis and cause a lesser degree of tissue reaction following placement. Suitable synthetic polymers include but are not limited to: Polyglactin 910; Poliglecaprone 25; Polysorb; Polydioxanone; Caprosyn; etc.

It will also be appreciated that the first component 110 can be formed of a non-absorbable material that is bio-compatible. Non-absorbable material is surgical material that is relatively unaffected by the biological activities of the body tissues and is therefore permanent unless removed. Bodies formed of non-absorbable material elicit a tissue reaction that results in encapsulation of the material by fibroblasts.

The non-absorbable material can be either of a monofilament construction or a multifilament construction. Suitable non-absorbable materials include monofilament or multifilament structures formed of natural and synthetic fibers or metal fibers, etc. For example, the non-absorbable material can be formed of natural material, including surgical silk; surgical cotton; surgical steel and synthetic materials including nylon; polyester fiber; polybutester; coated polybutester; polypropylene, etc.

As mentioned above, the first face 114 has a contoured or modified surface that intimately engages and is anchored to the target structure (e.g., the peritoneum). For example, the first face 114 can include a number of protrusions 120 that extend outwardly therefrom and are formed in select locations. The protrusions 120 can thus be in the form of teeth or barbs that extend outwardly from the first face 114. The protrusions 120 are constructed to provide members that easily embed within the target structure, such as the peritoneum 30, when a force is applied to the body 112 in a direction toward the target structure. The protrusions 120 can thus be pointed so as to promote being embedded within the peritoneum 30 when a sufficient force is applied to the body 112.

The protrusions (barbs) 120 can be formed in either a uniform manner across the first face 114 or they can be formed in a non-uniform manner. In addition, the protrusions 120 can be formed across the entire first face 114 or can be formed in one or more select locations, such as a central section of the first face 114 or along an annular pattern, or along and within a peripheral region, etc. Since the central section of the body 112 is the section that is intended for direct placement over the trocar entry site, it is preferred that this central section includes protrusions 120 to permit the body 112 to be securely attached to the target structure (e.g., peritoneum) in which the trocar entry site is formed. Thus, the body 112 is securely attached to the tissue/membrane that immediately surrounds the trocar entry site so as to provide a secure attachment of the body 112 to the target structure.

In one embodiment, the size of the body 112 is such that when it is applied to the target structure, the body 112 not only fully extends across and covers the trocar entry site but it also extends surrounds and extends a predetermined distance from the trocar entry site.

For example, in one embodiment, the body 112 has a dimension (e.g., width) that is at least two times the size (width) of the trocar instrument. For instance, when using a 10 mm sized trocar, the body 112 is sized to fully cover the trocar entry site and has a dimension (e.g., width) of at least 20 mm. However, it will be understood that the body 112 can have any number of different sizes relative to the trocar entry site. For example, for a 10 mm sized trocar, the body 112 can have a dimension (e.g., width) that is at least 30 mm or even at least 40 mm. In other words, the relative ratio of the size of the trocar entry site to the size of the body 112 is at least greater than 1:1; can be at least 1:2; can be at least 1:3; can be at least 1:4, etc.

The thickness of the body 112 is selected in view of the intended application and is sufficient enough to provide enough structure to the body 112 such that it can seat against the target structure (tissue/membrane) and close the trocar entry site and remain in a position against the target structure such that the trocar entry site is closed. The body 112 thus has sufficient rigidity to maintain its form (in at least a partially opened position) and be directed towards and into intimate contact with the target structure (e.g., peritoneum) such that the body 112 at least substantially maintains the opened position and extends across the trocar entry site and extend across the exposed surface of the target structure in areas surrounding the trocar entry site. It will be appreciated that the exposed surface of the surrounding areas around the trocar entry site represent a contact area between the body 112 and the target structure and is of sufficient area to allow for the intimate engagement between the body 112 and the target structure.

In the embodiment shown in FIGS. 1-10, the body 112 includes protrusions 120 that are formed along the peripheral edge thereof so as to form an annular shaped band of protrusions 120. However, the protrusions 120 can be uniformly formed across the entire surface (face 114) of the body 112. The protrusions 120 are in the form of pointed barbs.

The body 112 also includes a center hub 130 that provides a means for coupling the first component 110 to the second component 150. In this embodiment, the coupling means is in the form of a releasable mechanical attachment (friction fit) between the body 112 and the second component 150. The second component 150 can be in the form of an elongated structure that has a first distal end 152. For example, the second component 150 can be a rod-like structure with the proximal end defining a handle section for grasping by the user.

In one exemplary embodiment, the center hub 130 can include a male element 135 that is complementary and engages a corresponding female element 155 that is formed at the distal end 152. For example, the male element 135 can be in the form of a central boss that extends outwardly from the first face 114.

The shape and size of the boss 135 can vary depending upon the particular application. The boss 135 can thus be formed to have a square shape as shown; however, this is merely one exemplary shape. In addition, the height of the boss 135 should be such that it does not adversely impact the seating of the body 112 against the target structure (e.g. peritoneum).

The male element 135 can be formed of the same material that is used to form the body 112 or it can be formed of another similar material that is compatible for the intended application. For example, if the body 112 is formed of an absorbable material, the male element 135 should likewise be formed of an absorbable material. Similarly, if the body 112 is formed of a non-absorbable material, the male element 135 can likewise be formed of a non-absorbable material or can even be formed of an absorbable material.

In addition to the male element 135 itself, the body 112 can include a reinforced section immediately around the male element 135 to provide reinforcement around the male element 135. For example, in the illustrated embodiment, the reinforced area can have a disk-like appearance immediately around the male element 135. Since the male element 135 is part of the means for releasably coupling the body 112 to the second component 150, the reinforced section can provide additional integrity and support when a force is applied to the body 112 for disengaging it from the second component 150. The reinforced section can be formed in any number of different ways including simply forming the body 112 to have a greater thickness in this region or applying a coating or additional layer that is placed over the body 112.

The female element 155 is in the form of an opening that has a complementary size and shape relative to the male element 135. The female element 155 is thus formed and is open at the distal end 152. In the illustrated embodiment, the female element 155 is in the form of a square shaped opening that is of sufficient depth to fully receive the male element 135.

It will thus be appreciated that a mechanical attachment and in particular, a frictional fit is formed between the male element 135 and the female element 155 when the male element 135 is received within the female element 155. A snap fit can thus be formed between the male element 135 and the female element 155. When the second component 150 is mated to the body 112, the distal end 152 abuts the first face 114 or is located proximate thereto.

The mechanical attachment between the first and second components 110, 150 is such that when sufficient forces are applied in opposite directions, the second component 150 becomes disengaged from the first component 110, thereby permitting the first component 110 to remain in place against and securely attached to the target structures, such as the peritoneum 30 in the figures. For example, when a force is applied to the second component 150 in a direction away from the body 112 (e.g., in a direction orthogonal to the body 112) and a force is applied to the body 112 in a direction away from the second component 150, the coupling force (mechanical attachment (frictional fit)) is overcome resulting in the second component 150 disengaging from the first component 110. This allows the body 112 to remain in place and securely attached (e.g., anchored or embedded) to the target structure (e.g., peritoneum) such that the trocar entry site is covered (sealed) thereby preventing an object from entering the trocar entry site.

It will be appreciated that with respect to the device 100 and other devices described herein, the male and female mating features can be reversed in that with respect to the device 100, the male element 135 can be associated with the second component 150 and the female element 155 can be associated with the first component 110. The resulting mechanical attachment is the same as in the embodiments described herein.

As explained below, the second component 150 is sized so that it can travel freely within the bore 107 of the trocar 101 to allow both delivery to and retraction from the trocar entry site. For example, the diameter (width) of the second component 150 can be slightly less than the inner diameter of the trocar bore. In addition, the flexibility of the first component 110 permits it to be folded so that it can be received into and pass through the bore 107 of the trocar when attached to the second component 150.

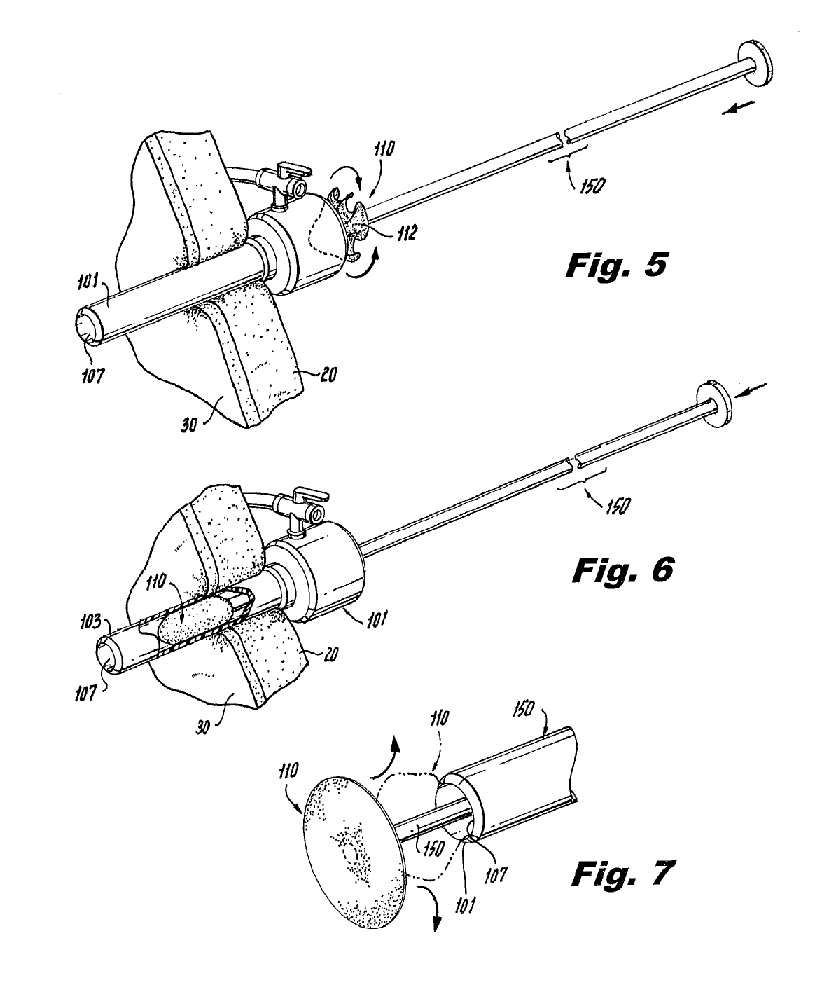

The method of closing the trocar site is now described with reference to FIGS. 4-10. FIG. 4 shows the trocar 101 already inserted into the abdominal cavity 10 such that it passes through the fascia 20 and the peritoneum 30. The insertion of the trocar 101 is performed by conventional surgical procedures. The first and second components 110, 150 are securely coupled to one another outside of the trocar 101 as shown in FIG. 5. In the embodiment of FIGS. 1-10, the first and second components 110, 150 are coupled to one another by inserting the male element 135 into the female element 155 of the second component 150. In other words, the body 112 is positioned at the distal end 152 of the second component 150 with the first face 114 thereof facing the distal end 152. The male element 135 thus faces the distal end 152. The male element 135 is then inserted into the female element 155 such that a mechanical fit results. For example, a frictional fit is formed between the boss 135 and the second component 150. Since the body 112 in its fully opened position is larger than the trocar 101, the body 112 is folded (e.g., folded upwardly) so as to allow insertion of the second component 150 within the bore 107 of the trocar 101. The folded body 112 thus surrounds the distal end 152 of the second component 150 and can slidingly travel within the bore 107 between the inner wall of the trocar 101 and the second component 150. FIGS. 5-6 show the folded body 112 being inserted and advanced within the bore 107 while maintaining its connection to the second component 150.

As shown, the distal end of the trocar 101 extends beyond the peritoneum 30 (target structure) and the bore 107 thus opens to the abdominal cavity 10. As shown in FIG. 7, the second component 150 is advanced within the bore 107 until the body 112 is advanced beyond the distal end of the trocar 101. Once the body 112 is free of the confines of the trocar 101, the body 112 will unfold into an at least partially open position in which the body 112 lies flat or lies approximately flat. It will be appreciated that the body 112 still remains securely attached to the second component 150 at this time.

It will also be appreciated that the trocar 101 itself can be used to at least partially open or more fully open the body 112 after it exits the trocar 101. For example, the body 112 partially opens once it extends beyond the distal end of the trocar 101. However, in order to ensure a more complete opening and to position the body 112 in a more planar position, the trocar 101 can be advanced over the second component 150 (which is held stationary) so as to bring the distal end of the trocar 101 into contact with the body 112 (e.g., the first face 114 thereof). The continued forward movement of the trocar 101 relative to the body 112 causes the body 112 to flatten out more (i.e., to more fully open). After performing this technique, the trocar 101 can then be pulled back.

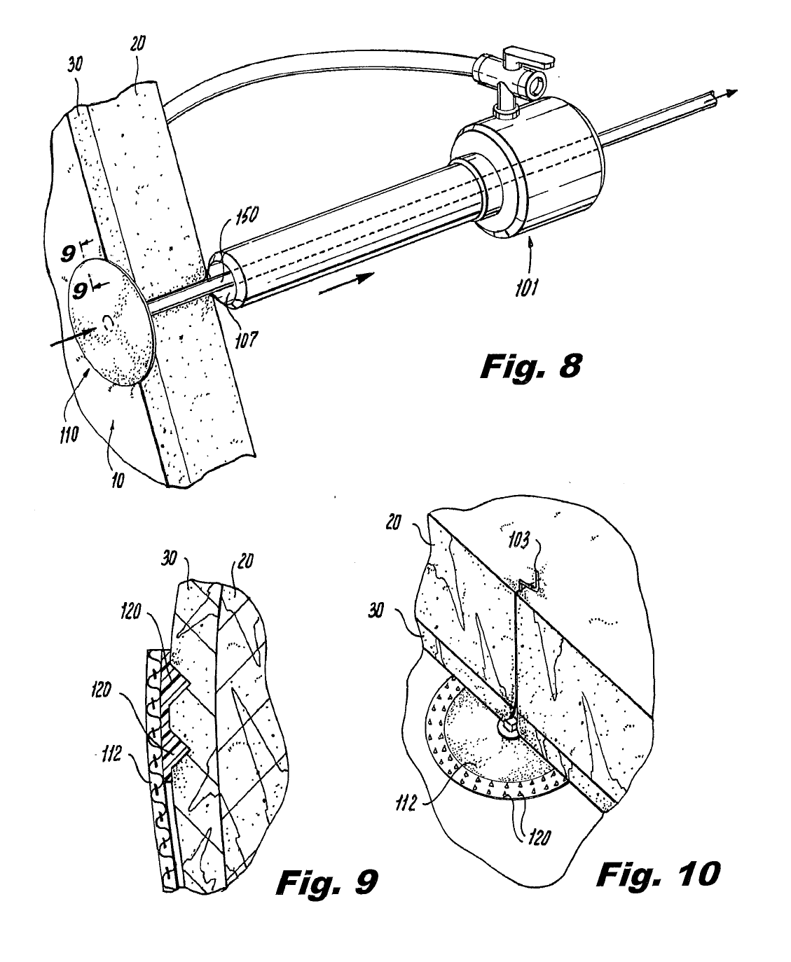

Next the first face 114 of the body 112 is moved in a direction toward the target structure (e.g., peritoneum) by pulling the second component 150 in a direction away from the abdominal cavity (i.e., in a direct away from the patient's body) as indicated by the arrow in FIG. 8. It will be appreciated that at the same time that the second component 150 is moved in this direction, the trocar 101 can be moved concurrently with the second component 150 in the same direction. As the second component 150 is moved in this direction, the body 112 likewise moves in the same direction towards the peritoneum 30. The continued pulling of the second component 150 causes the first face 114 of the body 112 to seat against the target structure, in this case, the peritoneum 30.

The protrusions 120 (barbs) serve as a means for securely attaching the body 112 to the target structure (peritoneum) by being at least partially embedded within the target structure as shown in FIG. 9. In other words, the pulling action of the second component 150 generates a force that drives the protrusions 120 into the target structure. Due to the material characteristics of the peritoneum 30, the protrusions 120 become at least partially embedded therein, thereby causing the body 112 to be securely attached to the peritoneum 30 in areas around the trocar entry site. This results in the trocar entry site being closed or shielded and objects, such as organs, within the abdominal cavity 10 cannot enter the trocar entry site.

The partially embedded/anchored body 112 is then disengaged from the second component 150. As described above, when a force is applied to the second component 150 in a direction away from the body 112 (e.g., in a direction orthogonal to the body 112) and a force is applied to the body 112 in a direction away from the second component 150, the coupling force (mechanical attachment (frictional fit)) is overcome resulting in the second component 150 disengaging from the first component 110. The target structure (peritoneum 30) acts as a stop and generates a force that is counter to the force generated by pulling the second component 150 in a direction away from the patient (and away from the body 112). As the second component 150 is thus continuously pulled in a direction away from the body 112, which is seated against the target structure (peritoneum), the frictional fit between the body 112 and the second component 150 is overcome, thereby resulting in the disengagement between the body 112 and the second component 150. The body 112 remains in place and securely attached to the peritoneum 30.

With the body 112 being disengaged, the second component 150 and the trocar 101 are then removed from the patient. Once the trocar 101 and second component 150 are removed, the incision can thus be closed along the surface of the skin as by using conventional sutures 103 or the like. This is shown in FIG. 10.

FIG. 13 also shows another means for securely attaching the body 112 to the target structure (peritoneum) besides protrusions (barbs) 120. The body 112 includes a band 119 that in the illustrated embodiment extends about the perimeter of the body 112 with the hub being centrally located. The band 119 is formed of a surgical grade adhesive that provides a sufficient bonding force to permit the body 112 to be driven into engagement with the peritoneum 30 resulting in the secure attachment of the body 112 to the peritoneum 30 due to an adhesive bond being formed.

FIGS. 14-15 illustrate a device 200 according to another embodiment. Device 200 is similar to device 100 and therefore, like reference legends are used for like elements.

The device 200 is formed of two separate components, namely, a first component 110 that is configured to remain at the surgical site and close the trocar site and a second component 210 that is an applicator or delivery device for delivering the first component 110 to the trocar site. The first component 110 can thus be generally thought of as being a patch-like member that covers and closes the trocar site without the use of sutures.

The first component 110 is similar to the one illustrated in FIGS. 1-10 with the main difference being the manner in which the second component 210 is securely attached to the first component 110. More specifically, FIGS. 14-15 show a different type of male-female mating relationship. The first component 110 includes a hub 121 formed on the first face 114 that includes the barbs 120. The hub 121 surrounds a center portion of the body 112.

As with the previous embodiment, the hub 121 can be formed of the same material or a different material relative to the body 112. The hub 121 is an upstanding continuous member that thus defines a center recessed portion 123 (e.g., a female element).

The second component 210 is an elongated instrument like the second component 150 and has a distal end 212. At the distal end 212, a flange 215 is provided. The flange 215 is outwardly directed and is formed of a flexible material. The flange 215 can thus be in the form of an 0 ring. The flange 215 is sized and shaped to be intimately received within and intimately engage and mate with the upstanding hub 121. In particular, the flange 215 sealingly mates with the upstanding hub 121 so as to form a frictional fit between the second component 210 and the body 112. When the flange 215 is inserted into the center recessed portion 123, it mates with the upstanding hub 121 and forms a friction fit between the body 112 and the second component 210.

The operation and delivery of the device 200 is essentially the same as the device 100 in that the body 112 is folded and then at least partially opens when the body 112 is advanced beyond the trocar 101. The disengagement of the body 112 can result from the same as described above with respect to the second component 150 moves and in particular a force is generated to overcome the frictional fit between the two components.

Optionally and as shown in FIG. 15, the second component 210 can include an active means for disengaging the body 112 from the distal end 212. For example, the second component 210 can be a tubular structure with the flange 215 formed at the distal end 212 and includes a movable pusher 230 that is disposed within the hollow interior of the second component 210. The second component 210 can thus have an internal bore that is open at the distal end 212 and the movable pusher 230 is disposed therein.

The movement of the pusher 230 is controlled by an actuator, such as a push button or slider, etc. The actuator is operatively connected to the pusher 230 such that the pusher 230 can move between a fully extended position in which the distal end of the pusher 230 extends beyond the distal end of the second component 210 and a fully retracted position in which the distal end of the second component 210 is disposed within the internal channel and spaced from or is at the distal end of the second component 210. This action is much like the action of a ball point pen.

As shown, the pusher 230 can terminate at its distal end in an enlarged body, such as a disk-shaped structure that is sized to be received within the recessed portion. In other words, the enlarged body is received between the upstanding hub 121.

When the pusher 230 is moved to the fully extended position, the pusher 230 is driven into contact with the first face 114 of the body 112 and supplies a force in a direction toward the body 112. This driving force is sufficient to overcome the frictional fit between the two components and results in the body 112 being disengaged from the second component 210. When the pusher 230 is used, the pusher 230 can be used to assist in disengaging the body 112 after it has seated against the target structure (peritoneum 30).

Since the second component 210 and trocar 101 are removed from the site, the pusher 230 can be left in the fully extended position.

As with the previous embodiment, the body 112 can be formed of an absorbable material, a non-absorbable material or even a combination thereof. Similarly, the protrusions 120 can be uniformly distributed across the first face 114 or only a section of the first face 114 contains the protrusions 120.

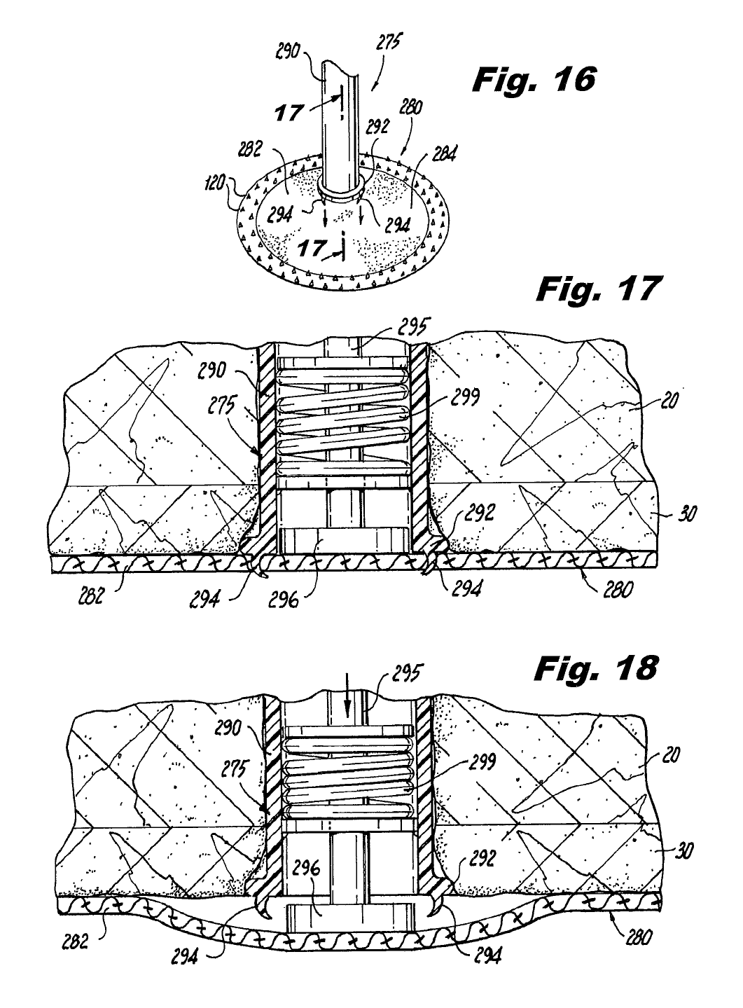

Now referring to FIGS. 16-18, a device 275 according to another embodiment is shown and is similar to the previous embodiments. In the device 275, a first component 280 has a body 282 that is formed in accordance with how the body 112 is formed. In other words, a first face 284 includes protrusions 120. The device also includes a second component 290 that is an elongated instrument that has a distal end 292. The distal end 292 includes at least one and preferably a plurality of sharp protrusions 294, such as teeth or barbs that extend outwardly from the distal end 292. As shown, the protrusions 294 can be inwardly curved teeth that are formed about a periphery of the second component 290. The teeth 294 are constructed and sized so as to produce a secure connection between the first and second components 280, 290 when the teeth 294 are at least partially embedded within the body 282. The body 282 is free of any hub.

In the illustrated embodiment, the second component 290 can be an at least partially or completely hollow member (e.g., a tubular structure) that includes a central bore 291. As shown in FIGS. 17-18, within the central bore 291 is an actuatable pusher 295 that moves in a linear manner within the central bore 291. For example, the pusher 295 can be biased within the central bore 291 as by a spring 299. The pusher 295 includes a shaft and an enlarged distal end section 296, such as a disc that fits within and can substantially occupy the central bore 291. The pusher 295 moves between a fully extended retracted position shown in FIG. 17 in which the distal end section 296 is disposed within the central bore 291 and does not extend beyond the distal end of the second component 290 and a fully extended position shown in FIG. 18 in which the distal end section 296 extends beyond the distal end of the second component 290.

As shown in FIG. 18, when the pusher 295 is advanced to the fully extended position, the pusher 295 serves as a means for disengaging the first component 280 from the second component 290. The pusher 295 is advanced a sufficient distance such that it is extended beyond the tips of the teeth 294 and thus causes the body 282 to locally be pushed forward away from the stationary teeth 294, thereby causing the body 282 to disengage from the teeth 294.

It will be appreciated that the operation of the pusher 295 occurs after the first component 280 is securely attached to the target structure (e.g., peritoneum) by means of the protrusions 120 that are embedded within the target structure. The pusher 295 thus does not interfere with the initial placement of the body 282 to the second component 290.

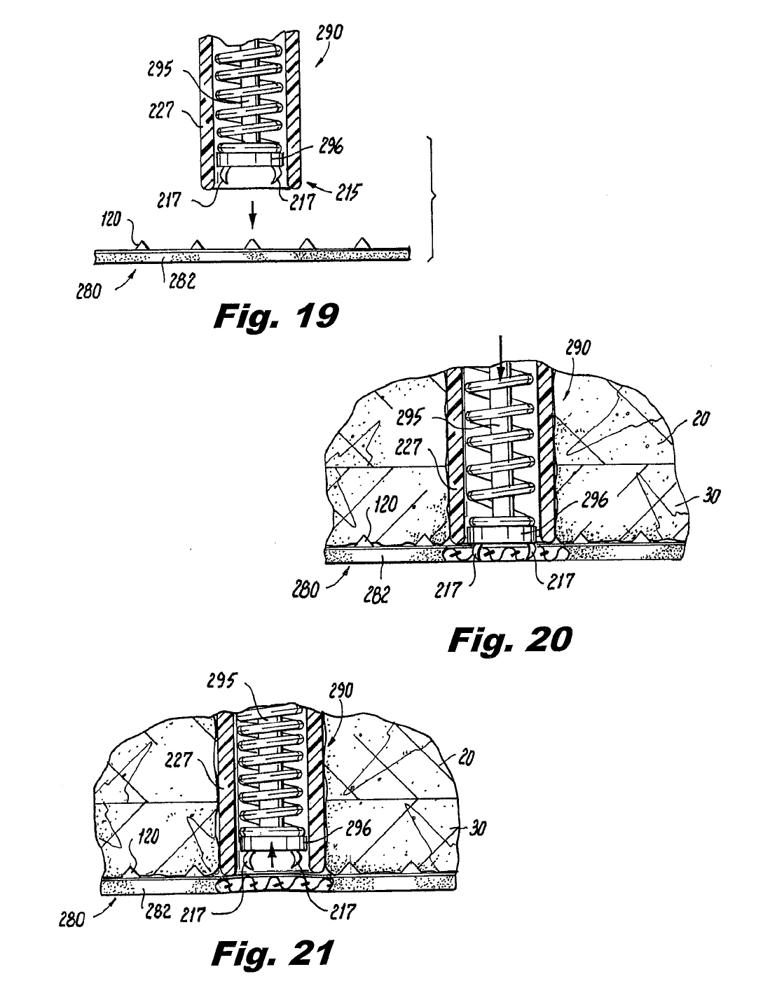

Now referring to FIGS. 19-21, in which a device 215 is shown. The device 215 is similar to the device 275 with the main difference being the location of the teeth. The device 215 includes a hollow tubular structure 227. In device 275, the teeth 294 are stationary, while in the device 215, the teeth 217 are disposed along an exposed, bottom surface of the pusher 295. Thus, when the pusher 295 is in the retracted position, the teeth 217 do not extend beyond the distal end of the second component (hollow tube 227). As a result, in the retracted position of the pusher 295, the teeth 217 do not intimately engage the body of the first component. The teeth 217 are inwardly directed, curved teeth.

FIG. 19 shows the first component 280 spaced from the second component 290 with the pusher 295 in the retracted position such that the teeth 217 at its distal end do not extend beyond the distal end of the second component 290. This is a pre-loaded position prior to attaching the first component 280 to the second component 290. FIG. 20 shows the body 282 in intimate engagement with the teeth 217 due to the pusher 295 being in the extended position and located distally beyond the distal end of the second component 290. In this position, the teeth 217 are at least partially embedded within the body 282. As in the other embodiments, when the first and second components 280, 290 are attached to one another, the body 282 can be partially folded to allow the insertion of the body 282 into the center bore of the second component 290 and then allow the body 282 to travel within the center bore to the open distal end where the body 282 exits and unfolds once it is free of the influence of the second component 290.

FIG. 20 shows the unfolded first component 280 having been pulling into engagement with the target structure (e.g., peritoneum) by means of the protrusions 120 (teeth/barbs). As in the other embodiments, the second component 290 is shown in the incision through which the trocar (not shown) passes to allow insertion of the first component 280 to the body cavity. The first component 280 has thus closed off the trocar entry site without the use of sutures along the target structure (e.g., peritoneum 30).

As in the other embodiments, after the first component 280 is securely attached to the target structure (e.g., peritoneum 30), the second component 290 is disengaged from the first component 280. In the embodiment of FIGS. 19-21, the second component 290 is disengaged from the body 282 by retracting the teeth 217 within the tubular structure of the second component 290, thereby disengaging the teeth 217 from the body 282. As shown in FIG. 21, as the pusher 295 retracts within the hollow interior of the second component 290, the portion of the body 282 to which the teeth 217 are attached is slightly drawn toward and even partially into the entrance into the trocar entry site (along the target structure (peritoneum 30)). The movement of the pusher 295 within the bore of the second component 290 in a direction away from the first component 280 overcomes the mechanical fit between the teeth 217 and the body 282.

Now referring to FIG. 23, a device 300 according to another embodiment is shown. The device 300 is similar to the previous devices and therefore, like elements are numbered alike.

The first face 114 of the body 112 includes a plurality of protrusions 120 and as with the previous embodiments, the protrusions 120 can extend across the entire first face 114 or only cover one or more portions thereof. In the illustrated embodiment, the protrusions 120 are in the form of an annular band or barbs; however, this is merely one embodiment.

The body 112 includes a plurality of pockets 160 that are formed about and at the periphery of the body 112. In the illustrated embodiment, there are three pockets 160 that are evenly spaced about the periphery (i.e., located 120 degrees apart). The pockets 160 are open along an edge that faces inwardly. Thus, all of openings of the pockets 160 face inward.

The device 300 includes a second component 310 that is similar to the other second components described herein and is in the form of an elongated member (e.g., a rod or shaft) that is gripped at a proximal end thereof. At a distal end 312 of the second component 310, there is a plurality of flexible fingers 315 that extend radially outward. The flexible fingers 315 can flex in multiple directions including a direction where the fingers 315 fold up toward the shaft of the second component 310 and the opposite direction where the fingers 315 fold downwardly away from the shaft. The direction of flexion depends upon the direction of the applied force.

The number of fingers 315 is equal to the number of pockets 160 to allow insertion of the fingers 315 within the pockets 160. In addition, the length of each finger 315 is selected such that when the finger 315 is in a substantially planar position (i.e., at a right angle to the shaft of the second component 310, the distal end 317 of the finger 315 is fully contained within the pocket 160 and is located proximate a closed end 162 of the pocket 160. As shown in FIG. 23, in the normal rest position, the fingers 315 lie generally perpendicular to the shaft of the second component 310 and therefore, when the fingers 315 are disposed within the pockets 160, the body 112 lies flat.

The insertion of the fingers 315 into the pockets 160 thus represents the mechanical attachment between the two components.

Before the device 300 is fed into the bore 107 of the trocar 101 (FIG. 6), the fingers 315 are disposed within the pockets 160 as by slightly flexing the fingers 315 to allow insertion of the fingers 315 within the pockets 160. Once all of the fingers 315 are disposed within the pockets 160, the body 112 is folded upward as in the other embodiments to allow insertion of the folded body 112 into the bore 107 of the trocar 101. In the folded position, the fingers 315 are folded upwardly.

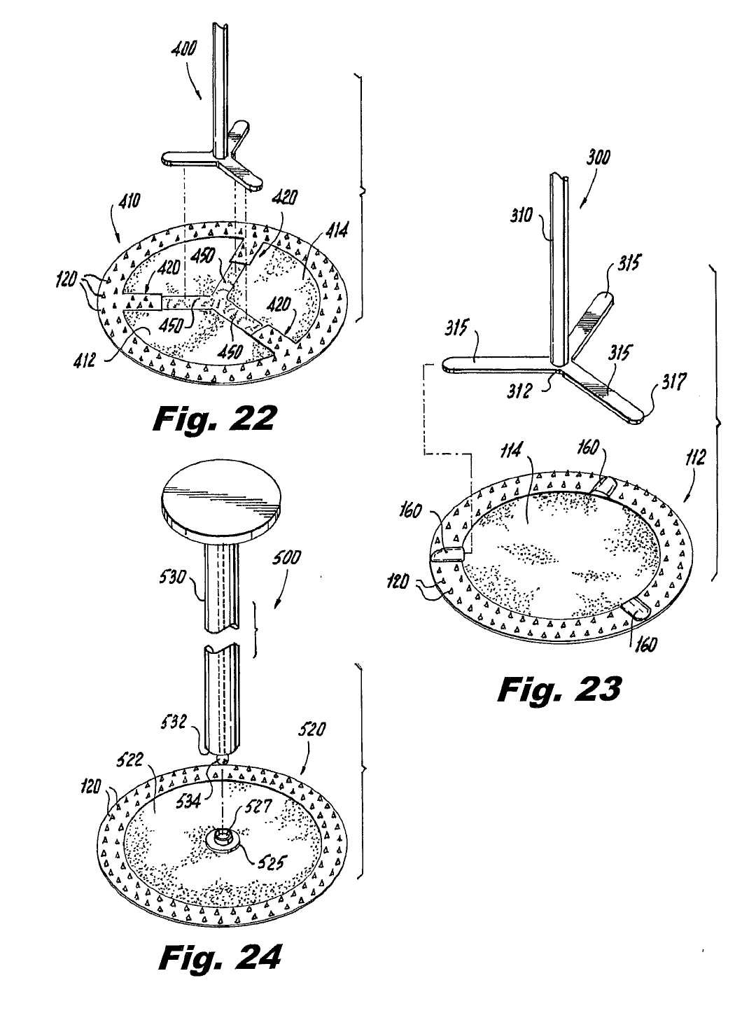

FIG. 22 illustrates another embodiment of a device 400 that is similar to the device 300. The device 400 includes the second component 310 with flexible fingers 315 formed at the distal end 312 thereof. The device 400 also includes a first component 410 that is similar to the first component 110 and includes a body 412 that can be constructed and include the same features as the body 112 described herein. Along a first face 414, there is a plurality of protrusions (e.g., barbs) 120 and a plurality of reinforcing elements 420 that are disposed along the first face 414. The reinforcing elements 420 provide local reinforcement of the body 412 at select areas, such as at three areas evenly spaced about the periphery of the illustrated embodiment. In other words, the three reinforcing elements 420 are located about 120 degrees apart.

The reinforcing elements 420 can be formed of the same material as the material that forms the body 412 or can be formed of other materials. For example, the reinforcing elements 420 can be a thicker region of the body or it can be a separate layer that is added to a homogenous body 412. The reinforcing elements 420 thus add additional integrity to the body 412 and assist in the natural movement of the body 412 to the open position.

In this embodiment, the means for attaching the second component 310 to the body 412 is in the form of adhesive bands 450 that are disposed along the first face 414. For example, the bands 450 can have a spoke-like pattern that are joined in the center of the body 412 and extend radially outward toward the peripheral edge. As shown, the bands 450 can extend toward and be disposed adjacent the inner edges of the reinforcing elements 420. The length of the fingers 315 in the device 400 is less than the length of the fingers 315 in the device 300 since the fingers 315 do not extend all the way to the peripheral edge and are not received within pockets, such as pockets 160. Instead, the fingers 315 are sized to seat against and intimately engage the adhesive bands 450 so as to provide a bond between the body 412 and the second component 310.

The bands 450 is formed of a surgical grade adhesive that provides a sufficient bonding force between the body 412 and the second component 310 to allow folding of the body 412, insertion and travel of the folded body 412 in the trocar bore 107 and unfolding of the body 412 once the body 412 is extended beyond the end of the trocar 101. The bonding force is also sufficient to permit the body 412 to be driven into engagement with the peritoneum 30 so as to at least partially embed the protrusions 120 therein resulting in the secure attachment of the body 412 to the peritoneum.

As in the other embodiments, the bonding force is overcome and the first component 410 is disengaged from the second component 310 by either continued pulling of the second component 310 in a direction away from the body 412 and/or initial forward advancement of the trocar 101 over the second component shaft, as described below, and then retraction of the trocar 101 and second component 310 in a direction away from the first component 410 (and away from the patient's body).

As in the other embodiments, the first component 410 is securely held to the target structure (e.g., peritoneum) by means of the protrusions 120.

FIG. 24 illustrates a device 500 according to yet another embodiment. The device 500 is similar to the other embodiments described herein and thus provides a device for closing a trocar site without the use of sutures. The device 500 includes a first component 520 that is similar to the other first components described herein and includes a hub 525 that can be formed identical or similar to how the other hubs described herein are formed. The hub 525 includes a female element 527 in the form of a socket. As in the previous embodiments, the hub 525 can be formed of the same material or a different material relative to the body 522 of the first component 520. The socket 527 can thus be a curved recessed portion (e.g., concave shape).

The body 522 includes protrusions 120 of the type described hereinebefore.

The device 500 also includes a second component 530 that is complementary to the first component 520 and is configured to securely attach thereto in a releasable manner. The second component 530 is similar to the other second components described herein and is in the form of an elongated instrument that has a proximal end for holding by the surgeon and a distal end 532. At the distal end 532, a male element 534 is provided and is complementary to the female element 527 such that when the two mate together, a secure mechanical (frictional) fit results. In the present embodiment, the male element 534 is in the form of a ball that mates with the socket 527 so as to provide a frictional fit between the two components (in a manner as to how traditional ball and socket parts work). The ball 534 is effectively captured within the female element 527 to form the friction fit.

The steps for securely attaching the body 522 to the target structure (e.g., peritoneum) is essentially the same as in the embodiments shown in FIGS. 1-10 in that the frictional fit between the ball 534 and the socket 527 is overcome by a pulling force applied to the shaft of the second component 530 is a direction away from the first component 520 after the body 522 has effectively been seated and securely attached to the target structure (e.g., peritoneum) as by at least partially embedding the protrusions 120 within the peritoneum or other structure. The seating and attachment of the peripheral portions of the body 522 to the peritoneum creates a stop and generates a counter force to the pulling force; however, when a sufficient pulling force is generated, it overcomes this counter force and results in the ball 534 disengaging from the socket 527.

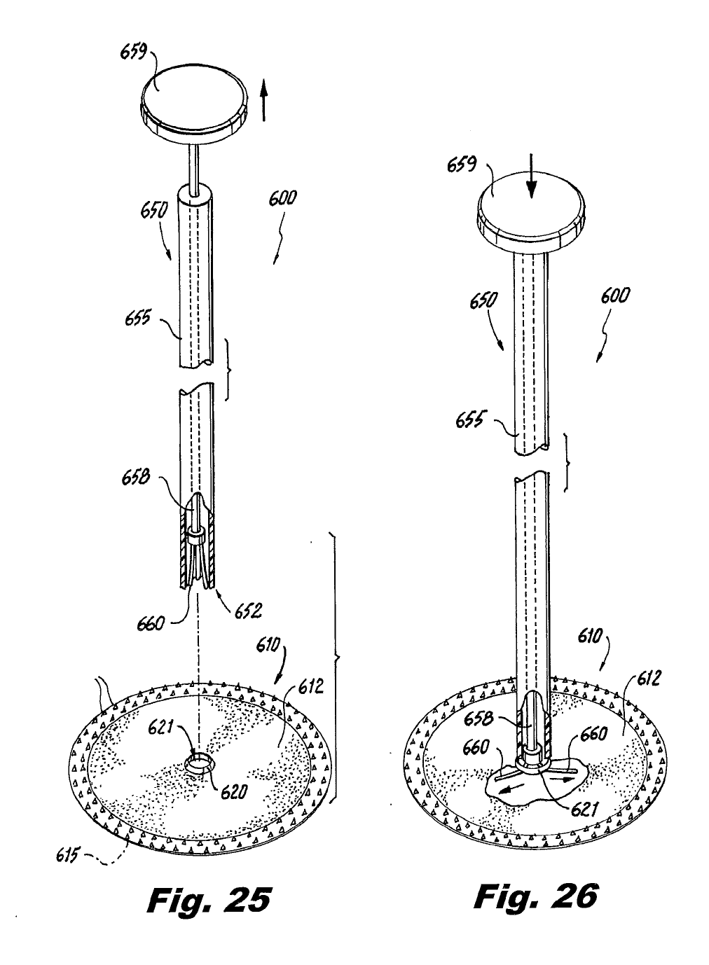

FIGS. 25-26 illustrate a device 600 according to yet another embodiment. As with the other embodiments, the device 600 is intended to close a trocar site without the use of sutures and includes a first component 610 that is similar to the first component 110 and is left at the trocar site in position such that it covers the trocar entry site and is securely attached to the target structure (e.g., peritoneum). Like the first component 110, the first component 610 is defined by a body 612 (e.g., formed of an absorbable material or a non-absorbable material or even a combination thereof) that has a first face 614. The first face 614 includes a plurality of protrusions 120, such as teeth or barbs, that are formed across at least a portion of the first face 614.

The body 612 also includes a through opening 620 that is formed therein and can be centrally located as shown. The opening 620 can be reinforced as by using a grommet or the like 621 to ensure the integrity of the body 612.

The device 600 includes a second component 650 that is configured to provide a mechanism or feature for securely mating with the body 612 to provide a secure attachment thereto. For example, the second component 650 can include a plurality of deployable fingers or claws 660 that can extend from a distal end 652 of the elongated shaft of the second component 650. The deployable fingers 660 are formed of a memory material such that in a normal rest position, the deployable fingers 660 are outwardly curled at their distal ends as shown in FIG. 26 which represents a normal rest position of the fingers 660. The deployable fingers 660 can thus represent outwardly curled fingers that are formed circumferentially.

The second component 650 can include a hollow sheath 655 that receives an movable inner shaft 658 that terminates at its distal end with the deployable fingers 660. As shown, the inner shaft 658 terminates at its proximal end with an enlarged handle section 659 which has dimensions greater than the bore of the hollow sheath 655 so as to restrict the degree of forward movement of the inner shaft 658. As shown in FIG. 26, when the inner shaft 658 is in the most forward position and abuts the proximal end of the sheath 655, the deployable fingers 660 are in the fully extended position and extend beyond the distal end of the sheath 655 resulting in the fingers 660 curling since no force is applied thereto to cause a flattening of the fingers 660.

The inner diameter of the bore of the sheath 655 is complementary to the diameter of the opening 620. Thus, when the second component 650 seats against the first face 614 of the body 612, the bore of the sheath 655 is axially aligned with the through opening 620 and when the deployable fingers 660 are in a retracted position, the fingers 660 are oriented generally parallel to one another and are oriented generally longitudinally. When the inner shaft 658 is forwardly advanced within the bore of the sheath 655, the fingers 660 are advanced beyond the distal end of the sheath 655. Since the sheath 655 no longer applies a force to the deployable fingers 660, the fingers 660 return to their normal rest positions which are outwardly curled positions as shown in FIG. 26. In this position, the outwardly curled position causes the fingers 660 to curl on the underside (second face 615) of the body 612 (e.g., into contact therewith) and this provides an interference fit with the body 612 such that the body 612 does not automatically disengage from the second component.

Any number of different materials can be used to form the fingers 660 so long as when in the extended position, the fingers 660 assume a distal curled appearance. For example, a shape memory alloy (SMA, smart metal, memory metal, memory alloy, etc.) is an alloy that “remembers” its original, cold forged shape. One of the commercial uses of shape memory alloy involves using the pseudo-elastic properties of the metal during the high temperature (austenitic) phase. Frames or supports can be made of a shape memory alloy as they can undergo large deformations in their high temperature state and then instantly revert back to their original shape when the stress is removed. This is the result of pseudoelasticity; the martensitic phase is generated by stressing the metal in the austenitic state and this martensite phase is capable of large strains. With the removal of the load, the martensite transforms back into the austenite phase and resumes its original shape. This property of the shape memory alloy allows the metal to be bent, twisted and pulled, before reforming its shape when released. Based on the foregoing, the fingers 660 can be formed from a shape memory alloy that has characteristics that permit the intended function to be performed.

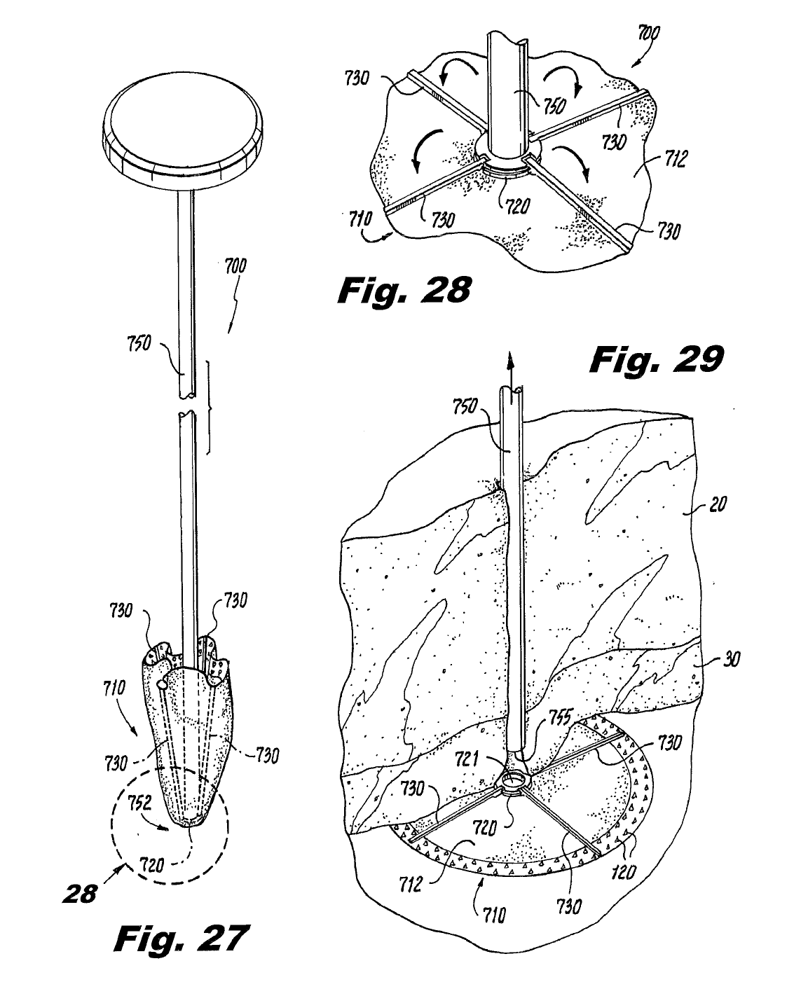

FIGS. 27-29 show a device 700 according to another embodiment. In this embodiment, the device 700 includes a first component 710 which can be the same as or similar to the first component 110 and include a means for securely, yet releasably, attaching the first component 710 to a second component 720. For example, any of the attachment means described herein can be used to attach the two components, including but not limited to a friction fit, deployable curled fingers, adhesive, etc.

In embodiment, the first component 710 includes a hub 720 that can be identical or similar to any of the hub structures described herein, such as hub 130, etc. The hub 720 further includes pivotable struts 730 that are pivotally attached at their proximal ends to the hub 720 so as to permit the struts 730 to pivot between open and closed positions. In the illustrated embodiment, the struts 730 are spaced about the circumference of the hub 720 and can be evenly distributed, such as at 90 degrees from one another. The distal ends of the struts 730 are securely attached to the body 712 of the first component 710. A mechanical or adhesive attachment can be provided.

In a normal rest position, the struts 730 lie flat across the body 712. The struts 730 thus can provide a weighting mechanism to ensure that the body 712 lies substantially flat.

For ease of illustration, the mechanical attachment between the first component 710 and a second component 750 is a ball and socket type. The second component 750 thus includes a ball (male element) 755 at the distal end 752. The hub 720 has a central recessed section 721 (i.e., a socket (female element)).

FIG. 27 shows the first and second components 710, 750 with the body 712 being folded upward and the struts 730 pivoted upward (similar to an umbrella). This is the position prior to insertion into the bore 107 of the trocar 101 and also during travel within the bore 107 of the trocar 101 to the trocar entry site so as to allow delivery of the body 712. FIG. 28 shows the opening of the body 712 that results when the body 712 is advanced beyond the distal end of the trocar 101. Once the body 712 is advanced beyond the trocar 101, such as into the abdominal cavity 10, the struts 730 facilitate the opening of the body 712 due to the pivoting movement.

It will be appreciated that the struts 730 can be formed of the same material or a different material relative to the material that forms the body 712.

FIG. 29 shows the open body 712 being seated against the target structure 30 (peritoneum). The protrusions 120 (barbs or teeth) serve as the means for securely attaching the body 712 to the target structure. As in the embodiment of FIGS. 1-10, once the body 712 is securely attached to the peritoneum 30 (or similar target structure), the shaft of the second component 750 is disengaged and removed from the first component 710 by passing through the trocar incision until the second component 750 exits the patient's body.

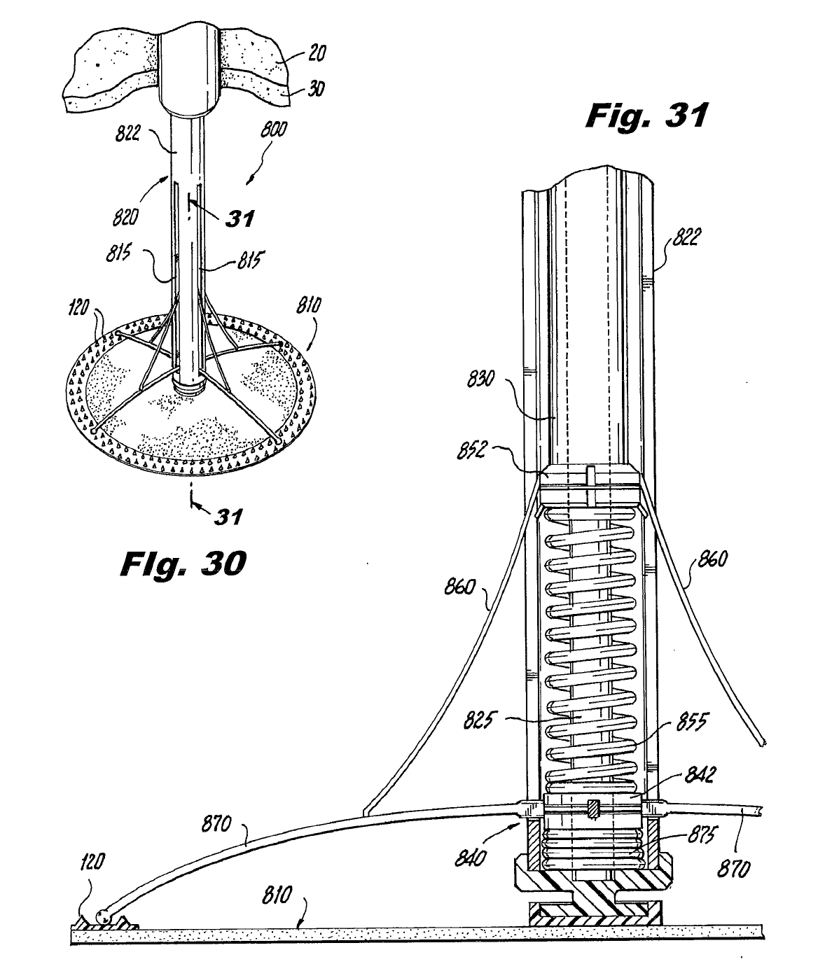

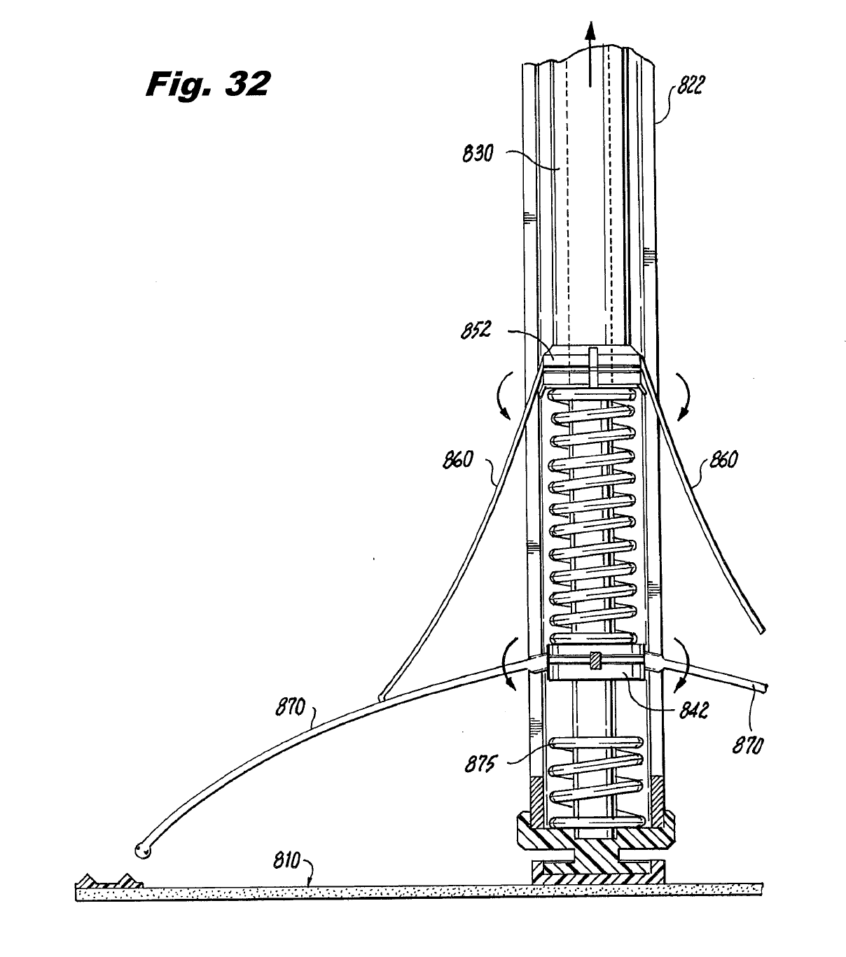

In yet another embodiment, the second component can utilize a passive means for securely attaching a distal end of the second component to the first component (patch body) and further includes an active mechanism to assist in unfolding the first component. This construction is shown in FIGS. 30-32. A device 800 includes a first component 810 which can be the same as or similar to the first component 110 and include a means for securely, yet releasably, attaching the first component 810 to a second component 820.

In this embodiment, the second component 820 includes an outer sheath 822 and an inner shaft 825 that is disposed within the sheath 822, as well as a movable shaft 830 that is longitudinally movable along the inner shaft 825 (e.g., along an exterior surface thereof). In particular, the outer sheath 822 can have a tubular construction, with an annular space being formed between the movable shaft 830 and the outer sheath 822.

The outer sheath 822 includes a plurality of notches 815 that are formed longitudinally along a length of the outer sheath 822 and are formed circumferentially about the sheath 822. The ends of the notches 815 can include cam surfaces (not shown), such as ramps, etc.

The movable shaft 830 is part of an active finger mechanism that includes a first finger assembly 840 and a strut assembly 850. The strut assembly 850 includes a strut carrier 852 that is fixedly attached to a distal end of the movable shaft 830. A plurality of struts 860 are provided and are pivotally attached to the strut carrier 852 to allow the struts 860 to move between extended positions (FIG. 30) in which the struts 860 extend outwardly from the strut carrier 852. The strut carrier 852 moves along the inner shaft 825. Similarly, the finger assembly 840 includes a finger carrier 842 and a plurality of fingers 870 that are pivotally attached to the finger carrier 842 to allow the fingers 870 to flex outward.

The finger carrier 842 is coupled to the strut carrier 852 by a means that permits the distance therebetween to be varied. For example, a biasing member 855, such as a spring, can be attached to an underside of the strut carrier 852 and to a top surface of the finger carrier 842. The biasing member 855 is disposed around the inner shaft 825. When the spring 855 is compressed, the distance between the carriers 842, 852 reduces.

A second biasing member 875 is disposed within the outer sheath 822 at a distal end thereof. The second biasing member 875 can be in the form of a spring that is disposed internally within the outer sheath 822 below the finger carrier 842. The second biasing member 875 is separate and not attached to the finger carrier 842; however, it applied a biasing force to the finger carrier 842.

The distal tips of the struts 860 are pivotally attached to the fingers 870 and are designed to assist in both the opening and closing of the fingers 870.

In the initial position prior to coupling of the first and second components, the struts 860 and fingers 870 are in the collapsed state and are located within the outer sheath 822. The struts 860 and fingers 870 can be located such that only distal portions of the fingers 870 are visible through the notches 815 or the fingers 870 are not visible through the notches 815 since they are located slightly above the top edge of the notch 815.

To extend the fingers 870, the movable shaft 830 is moved distally within the sheath 822, thereby causing the carriers 842, 852 to change position within the outer sheath 822. As the movable shaft 830 is driven downward, the distal tips of the fingers 870 encounter the notches 815 and the bowed shape and biasing characteristics of the fingers 870 cause the fingers 870 to flex outwardly. This causes the fingers 870 to pivot relative to the finger carrier. As the movable shaft 830 is continuously moved downward, more and more of the fingers 870 are exposed through the notches 815 and subsequently, the distal ends of the struts 860 encounter the notches 815 and flex outwardly therethrough. The struts 860 thus pivot open.

The continued driving of the movable shaft 830 causes the finger carrier to be driven into contact with the second biasing member 875, thereby causing compression thereof. As shown in FIG. 31, the second biasing member 875 compresses and stores energy. It will be appreciated that the movable shaft 830 can be locked in this position in which the fingers 870 are fully deployed. Alternatively, the movable shaft 830 cannot be locked and merely is pushed forward within the outer sheath 822 until the user feels resistance and compression of the second biasing member 875 which signals the fingers 870 are fully deployed.

In this fully deployed position, the struts 860 are also open. As the fingers 870 pivot open, the struts 860 pivot open.

As the fingers 870 open, the fingers 870 open and apply a force to the first component 810 for opening thereof. The fingers 870 are thus an active means for opening the first component 810. The fingers 870 are not directly attached to the first component 810 at the distal tips thereof.

As shown and similar to previous embodiments, the first and second components are detachably attached using a different means, such as a friction fit at a hub 811 that is part of the first component 810. In the illustrated embodiment, the second component includes a male component that is received within a female component that is part of the hub 811.

Once the first component 810 is opened, the user then retracts the second component by first retracting the fingers 870 and struts 860 before pulling the second component out of the trocar bore. To retract the fingers 870, the user pulls back on the movable shaft 830 and this results in a release of the stored energy of the second biasing member. A biasing force is applied to the underside of the finger carrier causing upward movement of the finger carrier. This upward applied biasing force causes the fingers 870 to pivot from the fully deployed position and in particular, as shown, the fingers 870 pivot downward. The firing of the second biasing member 875 thus causes the necessary movement of the finger carrier 852 that results in downward pivoting of the fingers 870.

The downward pivoting of the fingers 870 also results in downward pivoting of the struts 860. Continued pulling of the movable shaft causes retraction the fingers and struts; however, the first and second components remain attached. The fingers 870 and struts 860 can then fold back into the notches and back into the outer sheath 822.

It will be appreciated that the internal mechanism that controls movement of the fingers and struts can be any number of different types of mechanical linkages. For example, an actuator, such as a slider, can be provided and is connected to the struts by the mechanical linkage such that when the actuator is moved to an open position, the struts pivot open and outwardly. The fingers can thus be pivotally attached to the shaft of the second component and a biasing means can be used to open the struts.

Since the struts are integral to the second component, the struts are retracted prior to seating the open (unfolded) first component to the target structure (peritoneum). Thus, the internal mechanism has a means for retracting the struts.

It will be appreciated that the fingers 870 are opened once the folded (partially collapsed) first component advances beyond the distal end of the trocar and the distal end of the second component advances beyond the distal end of the trocar. The opening of the fingers 870 pushes the first component toward the fully opened position (flat position). Then the fingers 870 are retraceted leaving the first component in the opened position. The first component is still attached to the distal end of the second component by means of a mechanical attachment (e.g., male/female elements). The means of retracting the struts does not interfere with the attachment between the first and second components. Thus, the first and second components are disengaged by overcoming the mechanical attachment as by pulling the second component away from the first component as described herein when the first component seats against the target tissue. Since the fingers 870 and struts 860 have been retracted within the outer sheath 822, they do not interfere with the seating of the first component 810.

It will therefore be appreciated that the patch component (i.e., the first component of the embodiments described herein) is delivered through the trocar itself. The devices of the present invention can be utilized under direct vision and with experience can also be done in a blind manner. The roughened surface of the patch component allows it to be anchored into the peritoneal lining so that it “patches” the actual trocar hole, thus preventing any herination of bowel contents or other undesirable conditions when the patch is used in different settings.

As mentioned herein, the devices described herein can be used in a number of different surgical applications. For example, the device can be used to patch a trocar site in which a component (patch member) of the device is meant to cover intra-peritoneal lining and the hole created by trocar placement. The patch member indirectly covers the fascial lining but will not be primarily in contact with this area. In addition, the device can be used to patch areas in the body where NOS (natural orifice surgery) or NOTES is performed. NOTES surgery is becoming more popular and the ability to patch a small hole created during the NOTES procedure. One example is using an endoscope down the esophagus and into the stomach. A small hole is created in the stomach to enter the abdomen with the scope. Through the scope, a gallbladder or appendix can be removed. This hole created in the stomach can be patched with the present device. Another example, is performing a nephrectomy (kidney removal) or gallbladder removal through the vagina. The vaginal hole can be patched with the present device.

Another application for the present invention is to provide a patch of small defects in fascia or peritoneum. The present invention can be potentially used with patients with small hernias or defects in the abdomen of the patient. The hernia cannot be larger than the trocar (i.e., 20 mm maximum size). The present device can also be used as a patch in endoscopic cases. This use is similar to the NOTES application described above but it can be used in any application in which a scope is placed into the body and a small hole needs to be closed or patched. The device can also be used with fistulas or connections between different organs that are not “natural”. For instance, a colo-vesical fistula (connection between intestine and bladder) can be helped if a patch is placed between these organs.

In addition, while the devices are described as being used with a trocar, the devices of the present invention can be used with any number of different conduit/cannula structures that are introduced into the body and alternatively, in some specific settings, the instruments described herein may be introduced into the body without use of a trocar-like device.

In terms of adhesives described herein, there are many commercially available biodegradable adhesives. The material (mesh-like) of the patch can be impregnated or placed on one face of the patch body.

In addition, as mentioned above, in yet another embodiment, the body of the patch is formed of a biocompatible material that has both material characteristics and a modified surface structure that promotes attachment between the body and the target structure (tissue). For example, the patch body can be formed to include nanoscale hills and valleys on its surface, similar to the flexible nanopillars covering geckos' sticky toes so as to produce a “biorubbery” type body. The hills and valleys create a patterned interface that enhances the surface area of contact and thus, the overall strength of the adhesion. The surface area of the patch body can be coated with a material that promotes a stronger bond between select types of target structures and in particular, a thin layer of material (e.g., sugar-based adhesive material) can be added to enable a stronger bond to wet surfaces, such as the bladder, etc. When the patch body is applied, capillary forces pull tissue into spaces between the pillars and the glue adheres to tissue proteins. The patch can be biodegradable and dissolved over time. Even without the added adhesive material, capillary forces pull the tissue into spaces between the pillars resulting in attachment of the patch to the target structure (tissue). The type of patch can be coupled to the delivery instrument using any of the techniques described herein and can be deployed using any techniques described herein.

It will therefore be appreciated that the patch (mesh body) of the present invention can be used anywhere in the patient's body in which a small hole or connection between natural layers occurs. The connection can be of a naturally occurring type (i.e., fistula) or made by physician (e.g., as by placement of the trocar) is not relevant.

The devices and methods of the present invention thus provide a novel product and technique to prevent the development of incisional hernias in trocar sites and also prevent other undesirable conditions within the human body. In addition, the elimination of sutures provides a more efficient manner of closing an opening, such as a trocar opening that is formed during trocar placement.

While the invention has been described in connection with certain embodiments thereof, the invention is capable of being practiced in other forms and using other materials and structures. Accordingly, the invention is defined by the recitations in the claims appended hereto and equivalents thereof.

What is claimed is:

1. A device for closing an opening formed in a patient's body without the use of sutures comprising:
a patch formed of a flexible body having a first face, the body being foldable and can be positioned between an open position and an at least partially closed position, wherein the body has a plurality of sharp protrusions formed along the first face for securely attaching the flexible body to a target structure within the patient's body in which the opening is formed such that the flexible body extends across and closes the opening; and
an instrument for delivering the patch to an inner face of a structure in which the opening is formed, the instrument having a first member that mates with the patch for securely, yet releasably, attaching the patch to the instrument to allow delivery of the patch.

2. A device for closing a trocar site without the use of sutures comprising:
a patch formed of a flexible body having a first face, the body being foldable and can be positioned between an open position and an at least partially closed position, wherein the body has a plurality of sharp protrusions formed along the first face for securely attaching the flexible body to a target structure in which a trocar opening is formed such that the flexible body closes the trocar opening; and
an instrument for delivering the patch to the trocar site, the instrument having a first coupling member that mates with the patch for securely, yet releasably, attaching the patch to the instrument to allow delivery of the patch;
wherein the instrument and the patch are configured to be received within and be advanced through a hollow trocar for delivery to the trocar site for closing of the trocar opening due to the sharp protrusions being configured to be at least partially embedded within the target structure.

3. The device of claim 2, wherein the patch comprises a mesh-like structure that is formed of a bio-absorbable material.

4. The device of claim 2, wherein the patch comprises a mesh-like structure that is formed of a non-absorbable material.

5. The device of claim 2, wherein the sharp protrusions are formed along peripheral regions of the patch.

6. The device of claim 2, wherein the sharp protrusions comprise a plurality of barbs.

7. The device of claim 2, wherein the first coupling member is at least one of a male element and a female element and the patch includes a complementary second coupling member that comprises at least one of a female element and a male element.

8. The device of claim 7, wherein a friction fit is formed between the first and second coupling members.

9. The device of claim 7, wherein the second coupling member is formed as part of a hub that is located centrally within the body of the patch.

10. The device of claim 7, wherein the male element comprises a protrusion and the female element comprises an opening shaped so that a friction fit results when the protrusion is received within the opening.

11. The device of claim 7, wherein the male element is a ball and the female element is a complementary socket.

12. The device of claim 7, wherein the second coupling member is formed of the same material that forms the body of the patch.

13. The device of claim 7, wherein the first coupling member comprises a plurality of flexible fingers and the patch includes a plurality of pockets formed along a peripheral edge of the patch, wherein distal ends of the flexible fingers are received within the pockets for securely attaching the patch to the instrument.

14. The device of claim 7, wherein the first coupling member comprises a plurality of flexible fingers and the patch includes a plurality of sections that contain an adhesive, whereby the patch and the instrument are mated together by intimate contact between the fingers and the adhesive sections.

15. The device of claim 14, wherein the adhesive comprises a surgical grade adhesive.

16. The device of claim 2, wherein the first coupling member includes a plurality of fingers that are positionable between a retracted position and an extended position, wherein in the extended position, the fingers have curled distal ends, the patch including a through opening formed therein for receiving the fingers when they are in the retracted position, the opening being sized such that the fingers, in the extended position, cannot pass therethrough.

17. The device of claim 16, wherein the instrument includes an outer tubular member and an inner shaft that includes the fingers at a distal end thereof, the inner shaft being slidingly movable within a hollow interior of the outer tubular member.

18. The device of claim 16, wherein the fingers are formed of a shape memory alloy and are naturally outwardly curled.

19. The device of claim 2, wherein the first member is part of a passive mechanism for attaching the patch to the second component and further including an active mechanism that is part of at least one of the patch and the second component for unfolding the patch.

20. The device of claim 19, wherein the active mechanism comprises a plurality of pivotable struts that are coupled at proximal ends to a center hub that is part of the patch and coupled at distal ends to a periphery of the patch, the pivotable struts lying substantially flat when the patch is in the open position.

21. The device of claim 19, wherein the active mechanism comprises a plurality of pivotable struts that are part of the instrument and are positionable between a fully extended position and a fully retracted position, wherein in the fully extended position, the fingers exert an opening force against the patch and in the fully retracted position, the instrument can pass through the trocar.

22. The device of claim 2, wherein a width of the patch is at least twice as great as a diameter of the trocar.

23. The device of claim 2, wherein a width of the patch is at least three times greater than a diameter of the trocar.

24. The device of claim 2, wherein the first coupling member comprises at least one tooth that is disposed at a distal end of the instrument, the patch being securely attached to the instrument by at least partially embedding the tooth within the patch.

25. The device of claim 24, wherein the instrument further includes an internal pusher that is movable between a retracted position and an extended position in which the pusher contacts the first face of the patch and drives the patch to disengagement with the instrument.

26. The device of claim 25, wherein the instrument includes a hollow outer member open at both ends with the internal pusher being movable disposed within the hollow outer member.

27. The device of claim 2, wherein the instrument includes a hollow outer tube open at both ends and an internal pusher that is movable within the hollow outer tube between a retracted position and an extended position, wherein an exposed face of the pusher at a distal end thereof includes at least one tooth, the patch being securely attached to the instrument by at least partially embedding the tooth within the patch when the pusher is in the extended position.

28. The device of claim 2, wherein the patch has a shape selected from the group consisting of a square, rectangle, circle, oval, and triangle.

29. The device of claim 2, wherein the target structure comprises an intra-peritoneal lining and the patch is shaped and sized to cover the trocar opening formed by trocar placement.

30. The device of claim 2, wherein the target structure comprises at least one of fascia, peritoneum and abdomen tissue.

31. A device for closing a trocar site without the use of sutures comprising:
a patch formed of a flexible body having a first face, the body being foldable and can be positioned between an open position and an at least partially closed position, wherein the body has an adhesive disposed along the first face for securely attaching the flexible body to a target structure in which a trocar opening is formed such that the flexible body closes the trocar opening; and
an instrument for delivering the patch to the trocar site, the instrument having a first coupling member that mates with the patch for securely, yet releasably, attaching the patch to the instrument to allow delivery of the patch;
wherein the instrument and the patch are configured to be received within and be advanced through a hollow trocar for delivery to the trocar site for closing of the trocar opening due to intimate contact between the adhesive and the target structure.

32. A method for closing an opening formed in a patient's body comprising the steps of:
inserting a trocar through an incision that defines a trocar opening for placement of the trocar;
attaching a patch to a distal end of an instrument, the patch being formed of a flexible body having a first face, the body being foldable and can be positioned between an open position and an at least partially closed position, wherein the body has a plurality of sharp protrusions formed along the first face;
inserting the folded patch and instrument within a bore of the trocar;
advancing the patch and instrument within the bore of the trocar until the patch advances beyond a distal end of the trocar resulting in the folded patch at least partially opening, the first face of the patch facing an inner surface of a target structure through which the incision is formed;
retracting the instrument in a proximal direction away from the patient's body so as to draw the patch into contact with the inner surface of the target structure, whereby the protrusions are at least partially embedded within the inner surface of the target structure, thereby securely attaching the patch thereto such that it closes off the trocar opening along the inner surface of the target structure; and
disengaging the instrument from the patch, thereby leaving the patch in place covering the trocar opening.

33. A patch for closing an opening formed in a patient's body without the use of sutures comprising:
a flexible bio-compatible body having a first face, the body being foldable and can be positioned between an open position and an at least partially closed position, wherein the body has a plurality of sharp protrusions formed along the first face for securely attaching the flexible body to a target structure within the patient's body in which the opening is formed such that the flexible body extends across and closes the opening as a result of the sharp protrusions being at least partially embedded within the target structure.
